(19) United States
(12) Patent Application Publication
(10) Pub. No.: US 2023/0228701 A1
(43) Pub. Date: Jul. 20, 2023

(54) GAS SENSOR AND CASING FOR CONTAINING SENSOR ELEMENT

(71) Applicant: NGK INSULATORS, LTD., Nagoya-shi (JP)

(72) Inventors: Shintaro MAKI, Taketoyo-cho (JP); Kota KATAGIRI, Ginan-cho (JP); Kohei YAITA, Nagoya-shi (JP); Yuya SEIKE, Nagoya-shi (JP)

(21) Appl. No.: 18/082,461

(22) Filed: Dec. 15, 2022

(30) Foreign Application Priority Data
Jan. 14, 2022 (JP) 2022-004315

(51) Int. Cl. G01N 27/407; G01M 15/10; G01N 27/406

(52) U.S. Cl. CPC G01N 27/407; G01M 15/102; G01N 27/4062

(57) ABSTRACT

A gas sensor for sensing a predetermined gas component contained in a measurement gas includes: a sensor element including a sensing part on one end portion thereof; a casing in which the sensor element is contained and secured; and a connector disposed in the casing, wherein the casing includes: an outer tube including a main portion in which a reference gas is included and a sealing portion as an end portion with a diameter smaller than the main portion so that another end portion of the sensor element protrudes to the main portion, a rubber seal member fitted into the sealing portion to seal the outer tube, and a spacer intervening between the seal member and the connector, and the spacer includes: a resin first spacer contacting with the seal member and having higher heat resistance than the seal member and a ceramic second spacer contacting with the connector.

### CROSS-REFERENCE TO RELATED APPLICATION

The present application claims priority from Japanese application JP2022-004315, filed on Jan. 14, 2022, the contents of which is hereby incorporated by reference into this application.

### BACKGROUND OF THE INVENTION

### Field of the Invention

The present invention relates to a gas sensor and, in particular, to a structure of a casing for containing a sensor element.

### Description of the Background Art

As a device for measuring a concentration of a predetermined gas component in a measurement gas, such as a combustion gas and an exhaust gas from an internal combustion engine typified by an engine of a vehicle, a gas sensor including a sensor element formed of oxygen-ion conductive solid electrolyte ceramics, such as zirconia (ZrO₂), has been conventionally known.

As the gas sensor, a gas sensor having a configuration in which an elongated planar sensor element (detection element) including oxygen-ion conductive ceramics (e.g., yttria stabilized zirconia) as a main constituent material thereof is contained in a tubular containment member (casing) made of metal has widely been known (see Japanese Patent Application Laid-Open No. 2015-178988, for example). The gas sensor is attached along an exhaust path of the internal combustion engine, and is used to sense the predetermined gas component in the exhaust gas and to measure the concentration thereof.

One end portion of the casing has an opening, and a seal member made of rubber is fit into the opening. A protective cover through which the exhaust gas can enter and exit is attached to the other end portion of the casing. The sensor element is contained in the casing while a portion between the both end portions is sealed to be airtight. This allows one end portion of the sensor element to be in contact with a reference gas (typically, ambient air) in the casing on a side of the one end portion of the casing, and allows the other end portion of the sensor element to be exposed in the protective cover to be in contact with the exhaust gas on a side of the other end portion of the casing in the gas sensor. The reference gas and the exhaust gas are not in contact with each other.

The seal member made of rubber is fit into the opening of the casing after a lead for electrically connecting the sensor element to an outside is inserted into a through hole formed in advance, and the fit portion of the casing is swaged from a side part thereof together with the seal member to prevent ingress of water from outside through the opening.

The sensor element used for the gas sensor typically includes a heater for heating the oxygen-ion conductive ceramics to activate the oxygen-ion conductive ceramics. The gas sensor is thus at a high temperature when being in use not only due to heat transferred through piping and heat received from the exhaust gas generated with operation of the internal combustion engine but also due to heat generated by the heater of the gas sensor itself. Fluororubber, which is highly heat resistant, and the like are thus typically used for the seal member made of rubber.

There is a growing demand for shortening (reducing a length) of a gas sensor due to a narrowed component attachment space of the internal combustion engine in recent years. When responding to the demand by shortening the casing of the conventional gas sensor, the seal member made of rubber for closing the opening of the casing is brought close to piping or a heat source, such as the exhaust gas, in the piping. A gas sensor intended to deal with such a problem has been commonly known (see Japanese Patent Application Laid-Open No. 2005-227227, for example) In the gas sensor disclosed in Japanese Patent Application Laid-Open No. 2005-227227, a mica insulating member is sandwiched as a spacer between a seal member and a ceramic contact holding member (a separator in Japanese Patent Application Laid-Open No. 2005-227227), thereby suppressing thermal transmission to the seal member and prevent excessive increase in temperature of the seal member.

As disclosed in Japanese Patent Application Laid-Open No. 2005-227227, when the spacer intervenes between the rubber seal member and the ceramic separator to prevent the excessive increase in the temperature of the seal member, heat conductivity is preferably low from a viewpoint of suppressing the thermal transmission to the seal member. However, the spacer has a high temperature while the increase in the temperature of the seal member is suppressed, thus the spacer itself needs to have sufficient heat resistance.

### SUMMARY

The present invention is directed to a gas sensor and, in particular, to a structure of a casing for containing a sensor element.

According to the present invention, a gas sensor for sensing a predetermined gas component contained in a measurement gas, the gas sensor includes: a sensor element including a sensing part on one end portion thereof; a casing in which the sensor element is contained and secured; and a connector disposed in the casing to electrically connect the sensor element to an outside, wherein the casing includes: an outer tube including a main portion in which a reference gas is included and a sealing portion being an end portion having a smaller diameter than the main portion, another end portion of the sensor element protrudes to the main portion, a rubber seal member fitted into the sealing portion to seal the outer tube, and a spacer intervening between the seal member and the connector in the outer tube, and the spacer includes: a resin first spacer having contact with the seal member and having higher heat resistance than the seal member and a ceramic second spacer having contact with the connector.

According to the invention, increase in temperature of the seal member sealing the outer tube can be suppressed in the gas sensor, and heat resistance of the spacer itself can be ensured. Accordingly, thermal deterioration of the seal member can be suppressed while shortening the gas sensor more than a conventional configuration which does not include the spacer.

Accordingly, an object of the present invention is to provide a gas sensor including a spacer favorably achieving both suppression of increase in temperature of a seal member and ensuring of heat resistance, and capable of being shortened.

These and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a main-part cross-sectional view along a length of a gas sensor 100.

FIG. 2 is a diagram for description of a favorable dimensional relationship between a seal member 6 and a spacer 7.

FIG. 3 is a cross-sectional view along a length of a sensor element 10.

FIG. 4 is a diagram illustrating a comparison between configurations of four gas sensors.

### DESCRIPTION OF THE PREFERRED EMBODIMENTS

<Configuration of Gas Sensor>

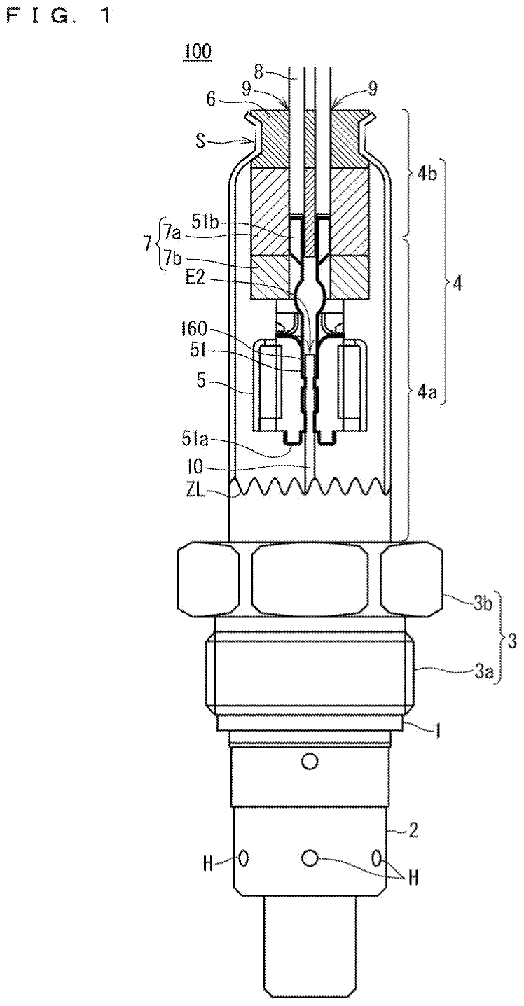

FIG. 1 is a partial cross-sectional view along a length of a gas sensor 100 (more particularly, a main body thereof) according to an embodiment of the present invention. More particularly, a cross-sectional view of the gas sensor 100 is illustrated above a break line ZL, and only appearance of the gas sensor 100 is illustrated below the break line ZL.

The gas sensor 100 is for detecting a predetermined gas component (e.g., NOx) using a sensor element 10 included therein. The gas sensor 100 generally has a configuration in which an elongated columnar or laminar sensor element (detection element) 10 is surrounded by a tubular body 1, a protective cover 2, a securing bolt 3, and an outer tube 4. The tubular body 1, the protective cover 2, and the outer tube 4 as a whole constitute a containment member (casing) for containing the sensor element 10 therein. On the other hand, the securing bolt 3 is fit around an outer side surface of the tubular body 1.

The sensor element 10 is disposed coaxially with the tubular body 1, the protective cover 2, the securing bolt 3, and the outer tube 4. A direction of extension of a central axis of the sensor element 10 is also referred to as an axial direction. In FIG. 1, the axial direction matches an up-down direction in FIG. 1.

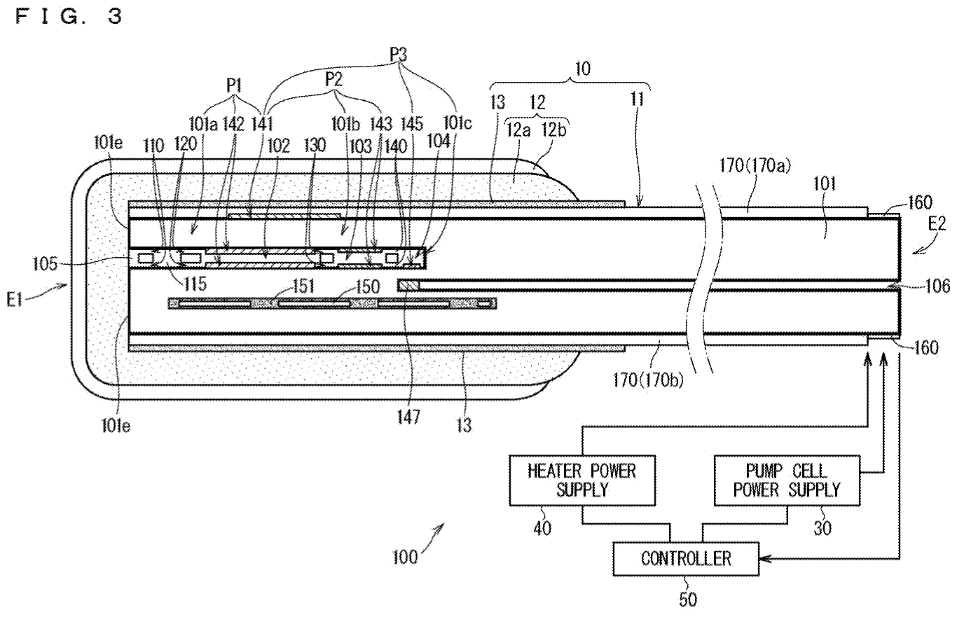

More particularly, one end portion (e.g., a first end portion El in FIG. 3) of the sensor element 10 is surrounded by the protective cover 2, the other end portion of the sensor element 10 protrudes into the outer tube 4, and a substantially middle portion between the end portions is secured in the tubular body 1 by an unillustrated ceramic green compact or ceramic component while being sealed to be airtight.

The sensor element 10 includes a sensing part (e.g., a gas inlet, an internal chamber, and a sensing electrode) on a side of the one end portion thereof surrounded by the protective cover 2. The sensor element 10 further includes various electrodes and wiring patterns on a surface of and in an element body thereof.

For example, in one aspect of the sensor element 10, a measurement gas introduced into the element is reduced or decomposed in the element to generate oxygen ions. The gas sensor 100 including the sensor element 10 having such a configuration determines the concentration of a gas component to be sensed in the measurement gas based on a quantity of oxygen ions flowing through the element proportional to the concentration of the gas component.

The tubular body 1 is a metal tubular member also referred to as a main metal fitting. The tubular body 1 is barely exposed to an outside of the gas sensor 100, and extends from an upper end portion in FIG. 1 of the protective cover 2 to a lower end portion in FIG. 1 of the outer tube 4. The sensor element 10 and a securing component (the ceramic green compact or the ceramic component) fit around the sensor element 10 are contained in the tubular body 1. In other words, the tubular body 1 is further fit around the fit component, which is fit around the sensor element 10. The protective cover 2 is a substantially cylindrical exterior member for protecting a predetermined range of the first end portion E1 of the sensor element 10 to be in direct contact with the measurement gas when being in use. The protective cover 2 is secured to a lower end portion in FIG. 1 of the tubular body 1 by welding.

The protective cover 2 has a plurality of through holes H through which gas can pass. The measurement gas flowing into the protective cover 2 through the through holes H is a direct sensing target of the sensor element 10. The types, the numbers, the locations, and the shapes of the through holes illustrated in FIG. 1 are just examples, and may be determined as appropriate in view of flow of measurement gas into the protective cover 2 into consideration.

The securing bolt 3 is an annular member used when the gas sensor 100 is secured to a measurement location. The securing bolt 3 includes a threaded bolt portion 3a and a holding portion 3b held when the bolt portion 3a is engaged. The bolt portion 3a engages with a nut disposed at an attachment location of the gas sensor 100. The gas sensor 100 is thereby secured at the measurement location with a side of the protective cover 2 thereof being in contact with a gas to be measured. For example, the bolt portion 3a engages with a nut portion disposed on an exhaust pipe of a vehicle so that the gas sensor 100 is secured to the exhaust pipe with the side of the protective cover 2 thereof being exposed in the exhaust pipe.

The outer tube 4 is a cylindrical member having one end portion (a lower end portion in FIG. 1) secured to an outer peripheral end portion of unillustrated upper side of the tubular body 1 by welding. The outer tube 4 includes a main portion 4a extending from a part where the outer tube 4 is secured to the tubular body 1 by welding to have a constant diameter in the axial direction, and a sealing portion 4b contiguous with the main portion 4a in the axial direction. The sealing portion 4b is an end portion having a smaller diameter than the main portion 4a.

An internal space of the outer tube 4 is a reference gas (ambient air) atmosphere. A connector (also referred to as a contact point holding member) 5 and the spacer 7 are disposed in the main portion 4a.

On the other hand, the sealing portion 4b is a portion laterally swaged with the seal member 6 being fit into the sealing portion 4b to seal the other end portion (an upper end portion in FIG. 1) of the outer tube 4.

The outer tube 4 is sealed by swaging an entire circumference of the sealing portion 4b from outside in a swaging portion S lateral to the seal member 6 in FIG. 1 so that the seal member 6 generates radially outward reaction force.

The seal member 6 is made of rubber. Thus, the seal member 6 is also referred to as a rubber plug. The rubber to be used is typically fluororubber. The seal member 6 has a uniform cylindrical shape before being fitted into the sealing portion 4b, but is deformed in a radial direction by fitting and swaging.

The other end portion (e.g., a second end portion E2 in FIG. 3) of the sensor element 10 is inserted into the connector 5. The connector 5 includes a plurality of contact point members 51 made of metal to be in contact with a plurality of electrode terminals 160 (see FIG. 3) of the sensor element 10 when the sensor element 10 is inserted. One end portion (a lower end portion in FIG. 1) of each of the contact point members 51 is a hooked portion 51a hooked to the connector 5, the other end portion (an upper end portion in FIG. 1) of each of the contact point members 51 is a crimping portion 51b to which a lead 8 is secured by crimping, and a portion between the end portions is a leaf spring portion. The contact point members 51 are secured by being sandwiched between the connector 5 and the sensor element 10, so that the electrode terminals 160 of the sensor element 10 and the contact point members 51 are electrically connected.

The spacer 7 is sandwiched (intervenes) between the connector 5 and the seal member 6 in the outer tube 4. The spacer 7 is provided to suppress increase in temperature of the seal member 6 in using the gas sensor 100. In the present embodiment, the spacer 7 has a two-tiered configuration of a first spacer 7a and a second spacer 7b. Details of the spacer 7 will be described later.

Each of leads 8 is inserted into through holes 9 sequentially provided in the seal member 6 and the spacer 7, and has one end portion secured to the crimping portion 51b of the contact point member 51 by crimping and the other end portion connected to a controller 50 and various power supplies (see FIG. 3) outside the gas sensor 100. The sensor element 10 is thereby electrically connected to the controller 50 and the various power supplies through the contact point members 51 and the leads 8. While only two contact point members 51 and two leads 8 are illustrated in FIG. 1, they are for ease of illustration, and the required number of leads for electrical connection described above are actually provided.

The gas sensor 100 having the above configuration can be manufactured by a method similar to a conventional method except for the intervention of the spacer 7 having the two-tiered configuration. Schematically, prior to swaging at the swaging portion S, the connector 5 into which the sensor element 10 has been inserted and in which the contact point members 51 have been connected to the leads 8d is disposed in the main portion 4a of the outer tube 4 in advance. The second spacer 7b, the first spacer 7a, and the seal member 6 are then stacked on the connector 5 in this order while the leads 8 are inserted into the through holes 9 therein. The seal member 6 into which the leads 8 are inserted is fitted into sealing portion 4b before swaging. Typically, ambient air as the reference gas has already entered the outer tube 4 before the seal member 6 is fit into sealing portion 4b. The swaging portion S is swaged by a predetermined swaging means after the seal member 6 is fitted.

It is a favorable example to swage the swaging portion S continuously extending over the outer periphery of sealing portion 4b, however, the swaging portion S may discontinuously extend in a circumferential direction of sealing portion 4b as long as a favorable swaging securing is achieved.

<Spacer>

Next, a configuration and a function of the spacer 7 are described in detail.

As described above, in the gas sensor 100, the spacer 7 has the two-tiered configuration of the first spacer 7a and the second spacer 7b, and as illustrated in FIG. 1, the seal member 6, the first spacer 7a, the second spacer 7b, and the connector 5 are disposed adjacent to each other in this order from the other end portion (an upper end portion in FIG. 1) of the outer tube 4.

Resin is selected as a material of the first spacer 7a from a viewpoint of having a low-thermal conductivity. Resin used for the first spacer 7a is preferably PTFE (polytetrafluoroethylene having a melting point of 327° C.) or PFA (perfluoroalkoxy alkane having a melting point of 310° C.), both of which are fluororesin. These types of resin have higher heat resistance than the rubber seal member 6 in addition to the low-thermal conductivity. For example, PTFE has a thermal conductivity of 0.2 W/m·K, and has a maximum continuous use temperature (a maximum temperature when use is continued at the maximum temperature) of 260° C.

In the meanwhile, ceramics having a higher melting point than resin is selected as a material of the second spacer 7b from a viewpoint of having better heat resistance than the first spacer 7a. Preferably selected is ceramics having a thermal conductivity of 32 W/m·K or less, which is also suitable from a viewpoint of heat insulating properties in addition to heat resistance. When ceramics satisfying such a range is selected as the material of the second spacer 7b, thermal transmission to the first spacer 7a and further to the seal member 6 is more preferably suppressed, and risk of thermal deterioration of the first spacer 7a and further the seal member 6 is further reduced. More preferably, alumina (thermal conductivity: 32 W/m·K) or steatite (thermal conductivity: 2 W/m·K) is selected.

That is to say, in the gas sensor 100 according to the present embodiment, the resin first spacer 7a having the low-thermal conductivity is adjacent to the seal member 6, and the ceramic second spacer 7b having the high heat resistance is adjacent to the first spacer 7a on a side opposite to the seal member 6.

As described above, the gas sensor 100 is secured at the measurement location with the portion on the side of the protective cover 2 thereof being in contact with the gas to be measured when being in use. In addition, as will be described below, the sensor element 10 includes a heater (e.g., a heater 150 in FIG. 3), and the sensor element 10 is sometimes heated by the heater when the gas sensor 100 is in use. That is to say, the gas sensor 100 is typically at a higher temperature in a lower portion in FIG. 1 when being in use. The configuration of intervention of the spacer 7 having the two-tiered configuration between the seal member 6 and the connector 5 as illustrated in FIG. 1 is intended to suppress the increase in temperature of the seal member 6 by the spacer 7 having heat resistance corresponding to such a temperature distribution.

More specifically, the rubber seal member 6 made of fluororubber, for example, has excellent adhesion to the outer tube 4, but is deteriorated when heated to a high temperature.

However, in the gas sensor 100 according to the present embodiment, the resin first spacer 7 a having the low-thermal conductivity is disposed adjacent to the seal member 6, and the ceramic second spacer 7 b having the high heat resistance is disposed on a side close to the sensor element 10, thus the heat resistance of the spacer 7 itself is ensured. Accordingly, even when the range from the protective cover 2 to the sensor element 10 has a high temperature when the gas sensor 100 is in use, preferably suppressed is excessive increase in temperature of the seal member 6 or occurrence of deformation and erosion of the spacer 7 and further the seal member 6.

In terms of PTFE as a preferred example of a material of the first spacer 7a, outgassing therefrom at a high temperature is low compared with rubber used for the seal member 6. Outgassing from the seal member 6 and the spacer 7 may cause contamination of reference gas (ambient air) in the casing and reduction in measurement accuracy of the gas sensor 100, however, according to the present embodiment, also achievable is a configuration of preferably suppressing outgas sing in outer tube 4 as well as the suppressing effect caused by suppressing the increase in the temperature of the seal member 6.

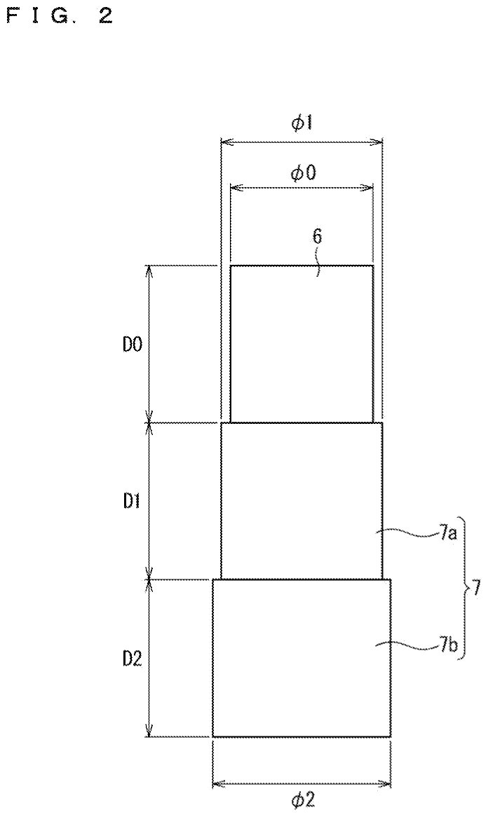

FIG. 2 is a diagram for description of a favorable dimensional relationship between the seal member 6 and the spacer 7.

It is assumed now that all of the seal member 6, the first spacer 7a, and the second spacer 7b have the cylindrical shape (in a state before fitted at least for the seal member 6), and as illustrated in FIG. 2, lengths (heights) in an axial direction are D0, D1, and D2, respectively, and outer diameters (diameters) thereof (in a state before fitted for the seal member 6) are φ0, φ1, and φ2, respectively.

Firstly, in terms of the length, a ratio D1/D2 is preferably 0.5 to 1.1. Such a range is satisfied, a heat resistance effect by the second spacer 7b is preferably obtained, and risk of thermal deterioration of the first spacer 7a and further the seal member 6 is reduced. If the length of the second spacer 7b is excessively short, the heat resistance effect by the second spacer 7b cannot be sufficiently obtained, and risk of thermal deterioration due to increase in temperature of the first spacer 7a and the seal member 6 is increased, thus is not preferable. If the length of the first spacer 7a is excessively short, the effect of suppressing thermal conductivity by the first spacer 7a cannot be sufficiently obtained, and risk of thermal deterioration due to increase in temperature of the seal member 6 is increased, thus is not preferable.

A range of preferable values of the lengths D0, D1, and D2 may be appropriately determined in accordance with a specific configuration and a shape of the gas sensor 100, and the length D0 is approximately a dozen mm to several tens of mm, and the lengths D1 and D2 are approximately several mm.

In the meanwhile, in terms of the outer diameter, a ratio φ1/φ0 is preferably equal to or larger than 0.95. When such a range is satisfied, the first spacer 7a has contact with substantially a whole surface of the seal member 6, thus thermal transmission from the first spacer 7a to the seal member 6 is uniformly performed, and occurrence of thermal deterioration of the seal member 6 caused by concentration of a portion of thermal transmission is preferably suppressed. If the outer diameter of the first spacer 7a is small and an area of contact with the seal member 6 is therefore small, thermal transmission occurs only in a contact position thereof, and thermal deterioration of the seal member 6 in the contact position as a starting point easily occurs, thus is not preferable.

The ratio φ1/φ0 is preferably equal to or smaller than 1.05. When such a range is satisfied, the second spacer 7b has contact with substantially the whole surface of the first spacer 7a, thus thermal transmission from the second spacer 7b to the first spacer 7a is uniformly performed on substantially the whole surface of the first spacer 7a, and occurrence of thermal deterioration of the first spacer 7a caused by concentration of a portion of thermal transmission is preferably suppressed. If the outer diameter of the first spacer 7a is large and an area of contact with the second spacer 7b is therefore small, thermal transmission occurs only in a contact position thereof, and thermal deterioration of the first spacer 7a in the contact position as a starting point easily occurs, thus is not preferable.

It is sufficient that a range of values of the outer diameters φ0, φ1, and φ2 is appropriately determined in accordance with a specific configuration and a shape of the gas sensor 100. Particularly, it is sufficient that the value of the outer diameter φ0 of the seal member 6 is slightly larger than an inner diameter of the sealing portion 4b of the outer tube 4, and the value of the outer diameters φ1 and φ2 of the first spacer 7a and the second spacer 7b are smaller than that of the main portion 4a of the outer tube 4 (that is to say, they do not have contact with the main portion 4a). For example, each of the outer diameters φ0, φ1, and φ2 is approximately a dozen mm to several tens of mm.

A configuration of intervention of the spacer 7 having the two-tiered configuration between the seal member 6 and the connector 5 adopted in the present embodiment is advantageous over the configuration in which the spacer 7 is not included, also in a viewpoint of shortening (reducing the length) of the gas sensor 100. The reason is that the spacer 7 can be disposed in a position closer to a high-temperature heating region (heat source) having a high temperature such that the seal member 6 is thermally5 deteriorated when the seal member 6 is solely disposed in the position as long as heat resistance of the second spacer 7b is ensured, and when the seal member 6 is disposed adjacent to the spacer 7, excessive increase in temperature of the seal member 6 is suppressed even if the arrangement position of the seal member 6 is located closer to the heat source than an allowable arrangement position in a case where the spacer 7 is not located. The state where the seal member 6 can be located close to the heat source indicates clearly that the gas sensor 100 can be shortened (reduced in length).

As described above, according to the present embodiment, the spacer intervenes between the connector disposed in the outer tube of the gas sensor to be connected to the sensor element and the seal member sealing the outer tube in the end portion of the outer tube, and the spacer has the two-tiered configuration of the resin first spacer and the ceramic second spacer adjacent to the first spacer, thus the increase in the temperature of the seal member can be suppressed, and the heat resistance of the spacer itself can be ensured. Accordingly, thermal deterioration of the seal member can be suppressed while shortening the gas sensor more than a conventional configuration which does not include the spacer.

<Example of Configuration of Sensor Element>

A configuration of the sensor element 10 for detecting NOx as an example of the sensor element 10 will finally be described. FIG. 3 is a cross-sectional view along the length of the sensor element 10 for detecting NOx. In this case, the sensor element 10 is a so-called limiting current type gas sensor element. FIG. 3 illustrates a pump cell power supply 30, a heater power supply 40, and the controller 50 of the gas sensor 100 in addition to the sensor element 10.

As illustrated in FIG. 3, the sensor element 10 generally has a configuration that a portion of an elongated planar element base 11 on the side of the first end portion El is covered with a porous leading-end protective layer 12. The element base 11 includes an elongated planar ceramic body 101 as a main structure, and main-surface protective layers 170 ( 170a and 170b) are arranged on two main surfaces of the ceramic body 101.

Furthermore, in the sensor element 10, the leading-end protective layer 12 (an inner leading-end protective layer 12a and an outer leading-end protective layer 12b) is disposed outside an end surface (a leading end surface 101 e of the ceramic body 101) and four side surfaces on a side of one leading end portion.

In the present embodiment, end portions of the ceramic body 101 and the sensor element 10 on the side of the first end portion E1 of the element base 11 are also referred to as first end portions E1, and end portions of the ceramic body 101 and the sensor element 10 on a side of the second end portion E2 of the element base 11 are also referred to as second end portions E2 for the sake of convenience.

The ceramic body 101 is made of ceramics including, as a main component, zirconia (yttrium stabilized zirconia), which is an oxygen-ion conductive solid electrolyte. The ceramic body 101 is dense and airtight.

The sensor element 10 illustrated in FIG. 3 is a so-called serial three-chamber structure type gas sensor element including a first internal chamber 102, a second internal chamber 103, and a third internal chamber 104 inside the ceramic body 101. That is to say, in the sensor element 10, the first internal chamber 102 communicates, through a first diffusion control part 110 and a second diffusion control part 120, with a gas inlet 105 opening to the outside on the side of the first end portion El of the ceramic body 101 (to be precise, communicating with the outside through the leading-end protective layer 12 ), the second internal chamber 103 communicates with the first internal chamber 102 through a third diffusion control part 130, and the third internal chamber 104 communicates with the second internal chamber 103 through a fourth diffusion control part 140, in outline. A path from the gas inlet 105 to the third internal chamber 104 is also referred to as a gas distribution part. In the sensor element 10 according to the present embodiment, the distribution part is provided straight along the length of the ceramic body 101.

The first diffusion control part 110, the second diffusion control part 120, the third diffusion control part 130, and the fourth diffusion control part 140 are each provided as two slits vertically arranged in FIG. 3. The first diffusion control part 110, the second diffusion control part 120, the third diffusion control part 130, and the fourth diffusion control part 140 provide predetermined diffusion resistance to the measurement gas passing therethrough. A buffer space 115 having an effect of buffering pulsation of the measurement gas is provided between the first diffusion control part 110 and the second diffusion control part 120.

An outer pump electrode 141 is provided on an outer surface of the ceramic body 101, and an inner pump electrode 142 is provided in the first internal chamber 102. Furthermore, an auxiliary pump electrode 143 is provided in the second internal chamber 103, and a measurement electrode 145 as the sensing part for directly sensing a gas component to be measured is provided in the third internal chamber 104. In addition, a reference gas inlet 106 which communicates with the outside and through which the reference gas is introduced is provided on the side of the second end portion E2 of the ceramic body 101, and a reference electrode 147 is provided in the reference gas inlet 106.

In the gas sensor 100 including the sensor element 10, the concentration of a NOx gas in the measurement gas is calculated by a process as described below.

First, the measurement gas flowing into the protective cover 2 through the through holes H and introduced into the first internal chamber 102 through the gas inlet 105 is adjusted to have an approximately constant oxygen concentration by a pumping action (pumping in or out of oxygen) of a main pump cell P1, and then introduced into the second internal chamber 103. The main pump cell P1 is an electrochemical pump cell including the outer pump electrode 141, the inner pump electrode 142, and a ceramic layer 101 a that is a portion of the ceramic body 101 present between these electrodes. In the second internal chamber 103, oxygen in the measurement gas is pumped out of the element by a pumping action of an auxiliary pump cell P2, which is also an electrochemical pump cell, so that the measurement gas is in a sufficiently low oxygen partial pressure state. The auxiliary pump cell P2 includes the outer pump electrode 141, the auxiliary pump electrode 143, and a ceramic layer 101 b that is a portion of the ceramic body 101 present between these electrodes.

The outer pump electrode 141, the inner pump electrode 142, and the auxiliary pump electrode 143 are each formed as a porous cermet electrode (e.g., a cermet electrode made of ZrO₂ and Pt that includes Au of 1%). The inner pump electrode 142 and the auxiliary pump electrode 143 to be in contact with the measurement gas are each formed using a material having weakened or no reducing ability with respect to a NOx component in the measurement gas.

NOx in the measurement gas caused by the auxiliary pump cell P2 to be in the low oxygen partial pressure state is introduced into the third internal chamber 104, and reduced or decomposed by the measurement electrode 145 provided in the third internal chamber 104. The measurement electrode 145 is a porous cermet electrode also functioning as a NOx reduction catalyst that reduces NOx existing in an atmosphere in the third internal chamber 104. During the reduction or decomposition, a potential difference between the measurement electrode 145 and the reference electrode 147 is maintained constant. Oxygen ions generated by the above-mentioned reduction or decomposition are pumped out of the element by a measurement pump cell P3. The measurement pump cell P3 includes the outer pump electrode 141, the measurement electrode 145, and a ceramic layer 101c that is a portion of the ceramic body 101 present between these electrodes. The measurement pump cell P3 is an electrochemical pump cell pumping out oxygen generated by decomposition of NOx in an atmosphere around the measurement electrode 145.

Pumping (pumping in or out of oxygen) of the main pump cell P1, the auxiliary pump cell P2, and the measurement pump cell P3 is achieved, under control performed by the controller 50, by the pump cell power supply (variable power supply) 30 applying voltage necessary for pumping across electrodes included in each of the pump cells. In a case of the measurement pump cell P3, a voltage is applied across the outer pump electrode 141 and the measurement electrode 145 so that the potential difference between the measurement electrode 145 and the reference electrode 147 is maintained at a predetermined value. The pump cell power supply 30 is typically provided for each pump cell.

The controller 50 detects a pump current Ip 2 flowing between the measurement electrode 145 and the outer pump electrode 141 in accordance with the amount of oxygen pumped out by the measurement pump cell P3, and calculates a NOx concentration in the measurement gas based on a linear relationship between a current value (NOx signal) of the pump current Ip 2 and the concentration of decomposed NOx.

The gas sensor 100 preferably includes a plurality of unillustrated electrochemical sensor cells sensing the potential difference between each pump electrode and the reference electrode 147, and each pump cell is controlled by the controller 50 based on a detected signal in each sensor cell.

In the sensor element 10, the heater 150 is buried in the ceramic body 101. The heater 150 is provided, below the gas distribution part in FIG. 3, over a range from the vicinity of the first end portion El to at least a location of formation of the measurement electrode 145 and the reference electrode 147. The heater 150 generates heat by being powered from the heater power supply 40 under control performed by the controller 50. The heater 150 is provided mainly to heat the sensor element 10 to enhance oxygen-ion conductivity of the solid electrolyte forming the ceramic body 101 when the sensor element 10 is in use. The sensor element 10 is heated so that the temperature at least in a range from the first internal chamber 102 to the second internal chamber 103 becomes 500° C. or more.

More specifically, the heater 150 is a resistance heating body made, for example, of platinum, and is provided to be surrounded by an insulating layer 151.

The plurality of electrode terminals 160 are formed on the respective main surfaces of the ceramic body 101 on the side of the second end portion E2 to establish electrical connection between the sensor element 10 and the outside. These electrode terminals 160 are electrically connected to the above-mentioned five electrodes, opposite ends of the heater 150, and unillustrated internal wiring for detecting heater resistance, through unillustrated internal wiring provided within the ceramic body 101 to have a predetermined correspondence relationship. As described above, the electrode terminals 160 are connected to the leads 8 via the contact point members 51, and application of a voltage from the pump cell power supply 30 to each pump cell of the sensor element 10 and heating using the heater 150 by being powered from the heater power supply 40 are thus performed through the leads 8, the contact point members 51, and the electrode terminals 160.

The main-surface protective layers 170 are layers made of alumina, having a thickness of approximately 5 μm to 30 μm, and including pores with a porosity of approximately 20% to 40%, and are provided to prevent adherence of any foreign matter and poisoning substances to the two main surfaces of the ceramic body 101 and the outer pump electrode 141. The main-surface protective layer 170 a thus functions as a pump electrode protective layer for protecting the outer pump electrode 141.

The leading-end protective layer 12 is provided around an outermost periphery of the element base 11 in a predetermined range from the first end portion El. The leading-end protective layer 12 is provided in a manner of surrounding a portion of the element base 11 in which the temperature is high (up to approximately 700° C. to 800° C.) when the gas sensor 100 is in use, in order to ensure water resistance in the portion to thereby suppress the occurrence of cracking (water-induced breakage) of the element base 11 due to thermal shock caused by local temperature reduction upon direct exposure of the portion to water.

In addition, the leading-end protective layer 12 is also provided to ensure poisoning resistance to prevent poisoning substance such as Mg from entering into the sensor element 10.

The inner leading-end protective layer 12a is made of alumina, has a porosity of 45% to 60%, and has a thickness of 450 μm to 650 μm. The outer leading-end protective layer 12b is made of alumina, has a porosity of 10% to 40%, which is lower than the porosity of the inner leading-end protective layer 12a, and has a thickness of 50 μm to 300 μm. The inner leading-end protective layer 12 a is provided as a low-thermal conductivity layer to have a function of suppressing thermal conduction from the outside to the element base 11.

The inner leading-end protective layer 12a and the outer leading-end protective layer 12b are formed by sequentially thermal spraying (plasma-spraying) constituent materials with respect to the element base 11 having a surface on which an underlying layer 13 has been formed.

As illustrated in FIG. 3, the underlying layer 13 is provided between the inner leading-end protective layer 12a and the element base 11 to secure an adhesion of the inner leading-end protective layer 12a. The underlying layer 13 is provided at least on the two main surfaces of the element base 11. The underlying layer 13 is made of alumina, has a porosity of 30% to 60%, and has a thickness of 15 μm to 50 μm.

<Modification>

While the limiting current type sensor element having three internal chambers and detecting NOx as a gas component to be detected is shown as an example of the sensor element 10 in the above-mentioned embodiment, the number of internal chambers may not be three and a gas component other than NOx may be detected in the sensor element 10 of the gas sensor 100. Alternatively, the sensor element may be a sensor element having no internal chambers, such as a mixed potential type sensor element.

### EXAMPLE

(Test 1: Heating Test)

The gas sensor 100 according to the above-mentioned embodiment was prepared as an example, and three gas sensors each having a configuration of the seal member and the spacer different from the gas sensor 100 were prepared as a comparative example 1 to a comparative example 3. A heating test was performed on these four gas sensors to compare a temperature of each seal member. The seal member 6 was made of fluororubber, the first spacer 7a was made of PTFE, and the second spacer 7b was made of alumina in the gas sensor 100. D0=6 mm, D1=6 mm, D2=6 mm, φ0=11 mm, φ1=11 mm, φ2=11 mm were applied.

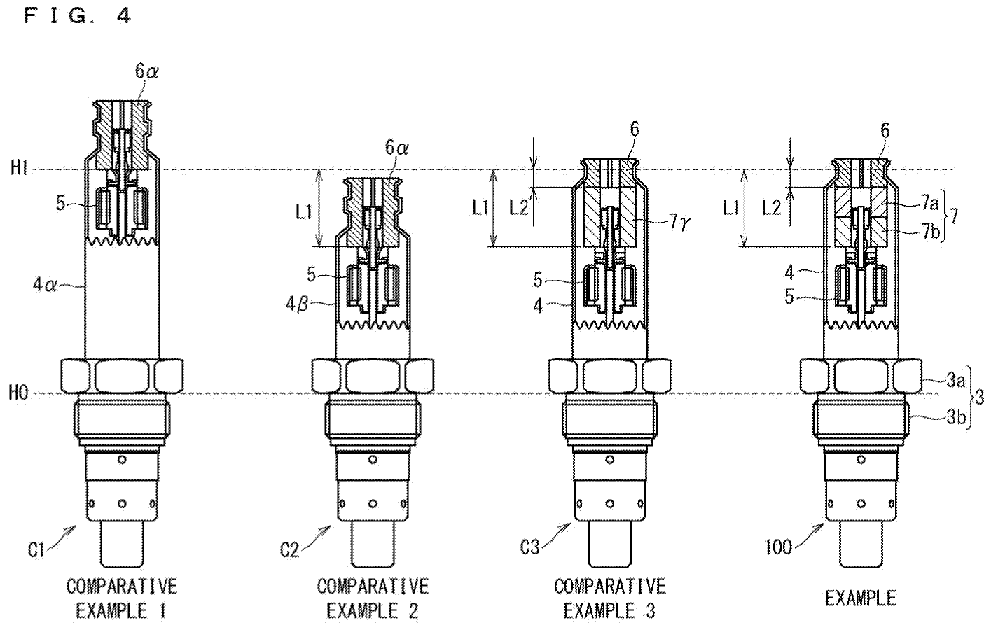

FIG. 4 is a diagram illustrating a comparison between configurations of four gas sensors as the gas sensor 100 according to the embodiment and the gas sensors C1 to C3 according to the comparative example 1 to the comparative example 3. In FIG. 4, a boundary position between the bolt portion 3a and the holding portion 3b of the securing bolt 3 in each of the four gas sensors is arranged at one height H0. A lower end position of a seal member 6α in the gas sensor C1 (a boundary position with an upper end of the connector 5) according to the comparative example 1 in the arrangement position is referred to as a seal member reference position H1.

The gas sensor C1 according to the comparative example 1 includes an outer tube 4a having a length longer than the outer tube 4 of the gas sensor 100 according to the example, and does not include a spacer. In the gas sensor C1, the seal member 6a has the length longer than the seal member 6 of the gas sensor 100, and a lower end of the seal member 6a has contact with an upper end of the connector 5 in the seal member reference position H1.

The gas sensor C2 according to the comparative example 2 has the same configuration as the gas sensor C1 according to the comparative example 1 except that the gas sensor C2 includes an outer tube 4β having a length shorter than the outer tube 4α of the gas sensor C1 according to the comparative example 1 (and furthermore, shorter than the outer tube 4 of the gas sensor 100 according to the example). In the gas sensor C2, a lower end position of the seal member 6α is located below the seal member reference position H1 by a distance L1 (=16 mm) in FIG. 4, and the lower end of the seal member 6α has contact with the upper end of the connector 5 in the lower end position.

In other words, in the gas sensor C2, reduction in length is achieved while maintaining the configuration except for the outer tube 4α in the gas sensor C1 as the elongated product of the conventional configuration described in the comparative example 1.

Furthermore, the gas sensor C3 according to the comparative example 3 has a configuration that a single spacer 7γ made of alumina intervenes between the seal member 6 and the connector 5 while achieving reduction in length in the manner similar to the comparative example 2. In the gas sensor C3, the lower end position of the seal member 6 is located below the seal member reference position H1 by a distance L2 (=4 mm) in FIG. 4, and the seal member 6 has contact with an upper end of the spacer 7γ in the lower end position. The spacer 7γ is located between the lower end position and a position below the seal member reference position H1 by the distance L1 in FIG. 4, and a lower end thereof has contact with the upper end of the connector 5.

The gas sensor C3 substantially has a configuration that a predetermined portion near the connector 5 of the seal member 6α in the gas sensor C2 according to the comparative example 2 is replaced with the spacer 7γ.

In the meanwhile, the gas sensor 100 according to the example includes the spacer 7 having the two-tiered configuration in place of the single spacer 7γ included in the gas sensor C3 of the comparative example 3. The gas sensor 100 has the configuration in common with the comparative example 3 in that the lower end position of the seal member 6 is located below the seal member reference position H1 by the distance L2 in FIG. 4, the seal member 6 has contact with the upper end of the spacer 7 in the lower end position, and the spacer 7 is located between the lower end position and the position below the seal member reference position H1 by the distance L1 in FIG. 4, and the lower end thereof has contact with the upper end of the connector 5, but different from the comparative example 3 in that the spacer 7 has a two-tiered configuration of the first spacer 7a made of PTFE and having contact with the lower end of the seal member 6 and the second spacer 7b made of alumina and having contact with the upper end of the connector 5.

The gas sensor 100 substantially has a configuration that a predetermined portion near the seal member 6 of the spacer 7γ in the gas sensor C3 according to the comparative example 3 is replaced with the resin first spacer 7a.

A heating test was performed on four gas sensors having the above-mentioned configurations to compare a temperature of each seal member. A C₃H₈ burner stand was used for heating, a gas temperature was set to approximately 770° C., and a lower side of the securing bolt 3 in FIG. 4 was heated. A heating time was 90 minutes, and the temperature was measured by a thermocouple. It is confirmed in advance that the seal member has a higher temperature as it gets closer to the lower side in FIG. 4 because of a configuration of heating facilities.

A result of the heating test is shown in Table 1 with distances from the seal member reference position H1 of the lower end of the seal member and the lower end of the spacer in each gas sensor. In Table 1, the seal member is referred to as “rubber plug”. In a column of “rubber plug temperature evaluation result” in Table 1, a degree of a temperature difference from a measurement temperature in the gas sensor C1 according to the comparative example 1 as a reference temperature and a measurement temperature in the other gas sensor is indicated with some marks. A circle mark indicates that the measurement temperature is lower than the reference temperature. A triangle mark indicates that the measurement temperature is equal to or higher than the reference temperature by less than 20° C. A cross mark indicates that the measurement temperature is higher than the reference temperature by 20° C. or more.

TABLE 1

| | DISTANCE | | DISTANCE | |
|---|---|---|---|---|
| | SHIFTED FROM | | SHIFTED FROM | |
| | REFERENCE | | REFERENCE | RUBBER PLUG |
| | POSITION OF END | | POSITION OF | TEMPERATURE |
| | PORTION OF | SPACER | LOWER END OF | EVALUATION |
| LEVEL | RUBBER PLUG | CONFIGURATION | SPACER | RESULT |
| COMPARATIVE | 0 mm | NONE | — | — |
| EXAMPLE 1 | | | | |
| COMPARATIVE | L1 = 16 mm | NONE | — | X |
| EXAMPLE 2 | | | | |
| COMPARATIVE | L2 = 4 mm | ALUMINA | L1 = 16 mm | Δ |
| EXAMPLE 3 | | | | |
| EXAMPLE 1 | L2 = 4 mm | PTFE/ALUMINA | L1 = 16 mm | ◯ |

First, it is confirmed from a result of the comparative example 2 that when the length is reduced while the configuration of the comparative example 1 is adopted, the seal member is heated to a higher temperature than the elongated product by 20° C. or more.

In contrast, in terms of the gas sensor C3 according to the comparative example 3 and the gas sensor 100 according to the example each having the configuration that a portion of the seal member 6a in the gas sensor C2 according to the comparative example 2 is replaced with the spacer, increase in temperature of the seal member is suppressed compared with the gas sensor C2 even though they are shortened products as with the gas sensor C2. This means that use of the ceramic spacer having excellent heat resistance has at least some degree of effect of suppressing the increase in the temperature of the seal member.

Particularly, in the case of the gas sensor 100 according to the embodiment in which the spacer 7 has the two-tiered configuration of the first spacer 7a made of resin and the second spacer 7b made of ceramics, the temperature of the seal member 6 is lower than the seal member 6a of the comparative example 1 even though the seal member 6 is located on the lower side of the seal member 6a in FIG. 4. This means that the configuration as with the above-mentioned embodiment that the spacer 7 has the two-tiered configuration, the resin first spacer 7a having the low-thermal conductivity is disposed on the side having contact with the seal member 6, and the ceramic second spacer 7b having the high heat resistance is disposed on the side having the high temperature when the gas sensor 100 is in use has particularly the effect of suppressing the increase in the temperature of the seal member 6.

The effect also suggests that the gas sensor 100 can be further shortened in the case of using the spacer 7 having the two-tiered configuration compared with the configuration illustrated in FIG. 4.

(Test 2: Thermal Deterioration Confirmation Test)

The gas sensor 100 according to the present embodiment and the gas sensor C2 according to the comparative example 2 were prepared, and the gas sensor 100 according to the embodiment was heated in a heating condition such that thermal deterioration (deformation) was intentionally generated in the seal member 6 of the gas sensor C2 to evaluate presence or absence of occurrence of the thermal deterioration. The C₃H₈ burner stand was used for heating in the manner similar to Test 1.

Specifically, a heating condition that the temperature of the seal member 6a of the gas sensor C2 became equal to or higher than 327° C. that is a melting point of PTFE was specified in advance, and the gas sensor 100 according to the embodiment was heated in the heating condition. In other words, the gas sensor 100 was heated in a heating condition such that thermal deterioration reliably occurred in the seal member 6a of the gas sensor C2. Such a heating condition is also considered a condition that thermal deterioration occurs in a spacer in a case where the spacer is made of only PTFE.

However, the thermal deterioration was not confirmed in any of the first spacer 7a made of PTFE and the seal member 6 included in the gas sensor 100. The above-mentioned result indicates that the ceramic second spacer 7b is provided, thus the heat resistance in the first spacer 7a and further the seal member 6 is ensured even in a high-temperature atmosphere in which PTFE is thermally deteriorated in a normal condition.

While the invention has been shown and described in detail, the foregoing description is in all aspects illustrative and not restrictive. It is therefore understood that numerous modifications and variations can be devised without departing from the scope of the invention.

What is claimed is:

1. A gas sensor for sensing a predetermined gas component contained in a measurement gas, the gas sensor comprising:
a sensor element including a sensing part on a side of one end portion thereof;
a casing in which the sensor element is contained and secured; and
a connector disposed in the casing to electrically connect the sensor element to an outside, wherein
the casing includes:
an outer tube including a main portion in which a reference gas is included and a sealing portion being an end portion having a smaller diameter than the main portion, another end portion of the sensor element protruding to the main portion,
a rubber seal member fitted into the sealing portion to seal the outer tube, and
a spacer intervening between the seal member and the connector in the outer tube, and
the spacer includes:
a resin first spacer having contact with the seal member and having higher heat resistance than the seal member and
a ceramic second spacer having contact with the connector.

2. The gas sensor according to claim 1, wherein
the first spacer is made of PTFE or PFA.

3. The gas sensor according to claim 1, wherein
he second spacer is made of ceramics having thermal conductivity of 32 W/m K or less.

4. The gas sensor according to claim 3, wherein
the second spacer is made of alumina or steatite.

5. The gas sensor according to claim 1, wherein
when lengths of the first spacer and the second spacer in an axial direction of the sensor element are D1 and D2, respectively,
a ratio D1/D2 is 0.5 to 1.1.

6. The gas sensor according to claim 1, wherein
when outer diameters of the seal member, the first spacer, and the second spacer are φ0, φ1, and φ2, respectively,
a ratio φ1/φ0 is equal to or larger than 0.95, and
a ratio φ1/φ2 is equal to or smaller than 1.05.

7. A sensor element containment casing for containing a sensor element and a connector while securing the sensor element therein, the sensor element including, on a side of one end portion thereof, a sensing part for sensing a predetermined gas component contained in a measurement gas, the connector electrically connecting the sensor element to an outside, the sensor element containment casing comprising:
an outer tube including a main portion in which a reference gas is included and a sealing portion being an end portion having a smaller diameter than the main portion, another end portion of the sensor element protruding to the main portion,
a rubber seal member fitted into the sealing portion to seal the outer tube, and
a spacer intervening between the seal member and the connector in the outer tube, wherein
the spacer includes:
a resin first spacer having contact with the seal member and having higher heat resistance than the seal member and
a ceramic second spacer having contact with the connector.

8. The sensor element containment casing according to claim 7, wherein
the first spacer is made of PTFE or PFA.

9. The sensor element containment casing according to claim 7, wherein
the second spacer is made of ceramics having thermal conductivity of 32 W/m K or less.

10. The sensor element containment casing according to claim 9, wherein
the second spacer is made of alumina or steatite.

11. The sensor element containment casing according to claim 7, wherein
when lengths of the first spacer and the second spacer in an axial direction of the sensor element are D1 and D2, respectively,
a ratio D1/D2 is 0.5 to 1.1.

12. The sensor element containment casing according to claim 7, wherein
when outer diameters of the seal member, the first spacer, and the second spacer are φ0, φ1, and φ2, respectively,
a ratio φ1/φ0 is equal to or larger than 0.95, and
a ratio φ1/φ2 is equal to or smaller than 1.05.

13. The gas sensor according to claim 2, wherein
the second spacer is made of ceramics having thermal conductivity of 32 W/m·K or less.

14. The gas sensor according to claim 2, wherein
when lengths of the first spacer and the second spacer in an axial direction of the sensor element are D1 and D2, respectively,
a ratio D1/D2 is 0.5 to 1.1.

15. The gas sensor according to claim 3, wherein
when lengths of the first spacer and the second spacer in an axial direction of the sensor element are D1 and D2, respectively,
a ratio D1/D2 is 0.5 to 1.1.

16. The gas sensor according to claim 2, wherein
when outer diameters of the seal member, the first spacer, and the second spacer are φ0, φ1, and φ2, respectively,
a ratio φ1/φ0 is equal to or larger than 0.95, and
a ratio φ1/φ2 is equal to or smaller than 1.05.

17. The sensor element containment casing according to claim 8, wherein
the second spacer is made of ceramics having thermal conductivity of 32 W/m·K or less.

18. The sensor element containment casing according to claim 8, wherein
when lengths of the first spacer and the second spacer in an axial direction of the sensor element are D1 and D2, respectively,
a ratio D1/D2 is 0.5 to 1.1.

19. The sensor element containment casing according to claim 9, wherein
when lengths of the first spacer and the second spacer in an axial direction of the sensor element are D1 and D2, respectively,
a ratio D1/D2 is 0.5 to 1.1.

20. The sensor element containment casing according to claim 8, wherein
when outer diameters of the seal member, the first spacer, and the second spacer are φ0, φ1, and φ2, respectively,
a ratio φ1/φ0 is equal to or larger than 0.95, and
a ratio φ1/φ2 is equal to or smaller than 1.05.
